(19) United States
(12) Patent Application Publication
(10) Pub. No.: US 2004/0039206 A1
(43) Pub. Date: Feb. 26, 2004

(54) Process for resolving racemic mixtures of piperidine derivatives

(76) Inventors: Eric David Moher, Greenwood, IN (US); Allie Edward Tripp, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/415,758

(22) PCT Filed: Nov. 13, 2001
(86) PCT No.: PCT/US01/42934

(51) Int. Cl. C07D 211/60

(52) U.S. Cl. 546/245

(57) ABSTRACT

A process for resolving a racemic mixture into its (R) and (S) enantiomers. The racemic mixture is reacted with a resolving agent selected from the group consisting of di-benzoyl-L-tartaric acid, di-benzoyl-D-tartaric acid, (S)-mandelic acid and (R)-mandelic acid in a solvent. The reaction is carried out under conditions sufficient to form soluble diastereomeric salts comprising the (R) enantiomer and the resolving agent, and the (S) enantiomer and the resolving agent, respectively. One of the diastereomeric salts is then isolated from the mixture. The free base of the isolated salt may then be generated in situ, and directly reacted with other compounds to synthesize useful chiral compounds.

The present invention relates to a process for resolving racemic mixtures. Specifically, the invention is a process to separate the enantiomers in a racemic mixture so that the resolved stereoisomers can be used in preparing chiral compounds.

It is well known in the pharmaceutical industry that enantiomers, or mirror image isomers, of a compound have biological activities that may differ markedly from each other. In order to optimize the desired activity, it is often beneficial to separate, or “resolve”, the enantiomers so that only the desired stereoisomer is administered. However, the commercial availability of suitable stereoisomer precursors is often limited. Even when such stereoisomer precursor compounds are available, their high cost may discourage widespread use.

Chiral compounds, such as ethyl nipecotate, are often useful as precursors in the synthesis of intermediates and useful compounds in the pharmaceutical industry. Although the racemic mixture is often utilized with favorable results in the formation of intermediates and compounds, in some instances it is desirable to utilize only the more active (R) or (S) enantiomer in order to synthesize substantially pure optically active pharmaceutical compounds. However, known resolution schemes for these compounds are often time consuming and require multiple operations. For example, according to one known process for generating the (S)-enantiomer of ethyl nipecotate from a racemic mixture, multiple crystallizations of diastereomeric salts derived from reaction with L-tartaric acid are required to effect the removal of the (R)-enantiomer. Following this, two more crystallizations with D-tartaric acid are required in order to generate the D-tartaric acid salt of (S)(+)ethyl nipecotate in 99% de and 32% overall yield, based upon 100% theory. See, Zheng, X.; Day, C.; Gollamudi, R.; Chirality, 1995, 7, 90, and references cited therein.

It would be beneficial to provide a simplified and straightforward process for resolving racemic lower alkyl nipecotate, which process is capable of providing resolved enantiomers at high yield and high enantiomeric purity.

The present invention provides a process for resolving a racemic mixture of compounds of Structural Formula I

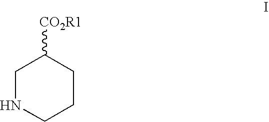

wherein R1 is C₁-C₄ alkyl or -N(R2)₂ where R2 is C₁ -C₄ alkyl, comprising:

(a) reacting a racemic mixture comprising (R) and (S) enantiomers of a compound of Structural Formula I and a resolving agent selected from the group consisting of di-benzoyl-L-tartaric acid, di-benzoyl-D-tartaric acid, (S)-mandelic acid and (R)-mandelic acid in a solvent under conditions suitable for forming soluble diastereomeric salts comprising said (R) enantiomer and said resolving agent, and said (S) enantiomer and said resolving agent, respectively;

(b) controlling said reaction conditions such that said diastereomeric salt comprising one of said (R) and (S) enantiomers separates from the mixture, wherein the diastereomeric salt comprising the other of said enantiomers remains in solution; and

(c) isolating the separated salt from said mixture.

The present invention, further, provides a process for resolving racemic ethyl nipecotate. A racemic mixture comprising the (R) and (S) enantiomers of ethyl nipecotate is reacted in a solvent with a resolving agent selected from the group consisting of di-benzoyl-L-tartaric acid and (S)-mandelic acid. The reaction is carried out at a temperature sufficient to form soluble diastereomeric salts comprising the (R) enantiomer and the resolving agent, and the (S) enantiomer and the resolving agent, respectively. The reaction mixture is cooled to a temperature such that the diastereomeric salt comprising the (S) enantiomer precipitates from the mixture, wherein the diastereomeric salt comprising the (R) enantiomer remains in solution. The precipitated salt comprising the (S) enantiomer is then isolated from the mixture.

### DETAILED DESCRIPTION OF THE INVENTION

The terms, abbreviations and definitions used in the present document have their normal meanings unless indicated otherwise. All expressions of concentration, percent, ratio and the like are expressed in weight units unless otherwise stated, except for mixtures of solvents which are expressed in volume units. Any temperatures not otherwise stated are expressed in degrees Celsius.

Chemical terms and the definitions below have their usual meanings unless indicated otherwise. For example, “N” refers to normal or normality; “mmol” refers to millimole or millimoles; “g” refers to gram or grams; “d” refers to density; “min” refers to minutes; “L” refers to liters; “mL” refers to milliliter or milliliters; “M” refers to mole or moles; “¹H-NMR” refers to proton Nuclear Magnetic resonance; “¹³C-NMR” refers to carbon-13 Nuclear Magnetic Resonance; “TLC” refers to thin layer chromatography; “HPLC” refers to high performance liquid chromatography; and “GC” refers to gas chromatography.

The term “enantiomer” is used to describe one of a pair of isomers that are mirror-images of each other and are non-superimposable.

The term “diastereomer” is used to describe a salt of an enantiomer.

The terms “racemic mixture”, “racemic compound” and “racemate” are used to describe mixtures of a compound comprising (R) and (S) enantiomers.

The term “enantiomeric excess” is used to describe the relative amount of an enantiomer in a racemic mixture. The enantiomeric excess of a particular enantiomer may be determined by calculating the amount of an individual enantiomer as a percentage of the entire mixture by the equation {(E1−E2)÷(E1+E2)}×100%=ee where E1 and E2 are the amounts of each individual enantiomer.

The term “diastereomeric excess” is used to describe the relative amount of a particular salt of an enantiomer (diastereomer) in a mixture of diastereomers. The diastereomeric excess of a particular diasteromer may be determined by calculating the amount of an individual diastereomer as a percentage of the diasteromeric mixture by the equation noted for enantomeric excess.

The abbreviation “ee” is used to describe enantiomeric excess.

The abbreviation “de” is used to describe diastereomeric excess.

The term “sustantially pure” is used to describe enantiomeric or diastereomeric purity of a single enantiomer or diastereomer which is greater than or equal to 90%, preferably greater than 95%.

The term “lower alkyl” is used to describe a C₁-C₄ straight or branched chain alkyl group. Examples of lower alkyl include methyl, ethyl, isopropyl, n-propyl, n-butyl, iso-butyl and tert-butyl.

The present invention describes a process for resolving a racemic mixture. The invention further describes novel diastereomer compounds. The invention also describes a process for preparing certain chiral compounds.

Suitable solvents for use in the resolution reaction include solvents in which the racemic compound and the resolving agent are substantially soluble at the reaction temperature. Non-limiting examples of suitable solvents are conventional organic solvents such ethyl acetate, isopropyl acetate, butyl acetate, acetone, acetonitrile, methyl tert-butyl ether (MTBE), tetrahydrofuran, 1,4-dioxane, diethyl ether and C₁ to C₄ alcohols, toluene, as well as water and mixtures of the foregoing. A particularly preferred solvent for resolving racemic ethyl nipecotate is ethanol. The ethanol can be absolute (99.5% or higher ethanol), or 95% denatured with toluene methanol isopropanol or mixtures of said denaturing agents. Use of an ethanol-based solvent hinders a transesterification reaction that might otherwise occur with the ethyl functional group of the ethyl nipecotate ester.

The use of an anti-solvent may be advantageous. As used herein, the term “anti-solvent” refers to a solvent in which the salt is significantly less soluble when compared to the solvent. Preferably, when an anti-solvent is used it is miscible with the selected solvent. Suitable anti-solvents include alkanes, such as pentane, hexane, heptane, cyclohexane, and the like. When the present process is carried out by crystallizing the acid addition salt from the racemic mixture, care must be taken in using an anti-solvent to avoid crystallization of the undesired diastereomeric salt.

Suitable resolving agents for use in the resolution reaction include agents capable of forming diastereomeric salts with each of the (R) and (S) enantiomers of the racemic mixture. The skilled artisan will appreciate diastereomeric salts precipitate from the solvent at different rates. Preferred resolving agents for use in resolving racemic ethyl nipecotate are di-benzoyl-L-tartaric acid, di-benzoyl-D-tartaric acid, (S)-mandelic acid and (R)-mandelic acid, respectively. Particularly preferred are di-benzoyl-L-tartaric acid and (S)-mandelic acid, and most preferred is di-benzoyl-L-tartaric acid. Typically the stoichiometry of the resolving agent in relation to the lower alkyl nipecotate ranges from about 0.1 to 1 equivalent, preferably from about 0.25 to 1 equivalent.

The inventive process may be carried out at temperatures ranging from about −20° C. to reflux. Those skilled in the art will appreciate that the reaction temperature for forming the diastereomeric salts should be high enough to enable the racemic mixture and the resolving agent to be substantially dissolved in the solvent, and to allow the reaction to proceed to equilibrium in a reasonable amount of time. Preferably, the reaction is carried out at an elevated temperature from of about 40° C. to about reflux. After the reaction is substantially completed, the reaction mixture is allowed to slowly cool to a temperature of from about ambient (room) temperature to about −20° C. As the solution cools, one of the diastereomeric salts precipitates from the mixture, while the other diastereomeric salt remains in solution. It may be advantageous to seed the solution as it cools. The precipitate, comprising a diastereomeric salt including one of the enantiomers, is then removed by conventional means, such as filtration, centrifugation, decanting, evaporation, drying and the like.

Although it is preferred to carry out the reaction at elevated temperature, the reaction may be carried out at room temperature. In this event, one of the diastereomeric salts forms a slurry in the solution, rather than a precipitate as before, while the remaining salt remains in solution. The slurry may be separated from the solution by conventional means.

The inventive method is particularly beneficial for the resolution of racemic nipecotate esters and amides. Nipecotate esters and amides, such as ethyl nipecotate, are precursors in the formation of intermediates for useful pharmaceutical compounds. Racemic nipecotate esters and amides are represented by Structural Formula I:

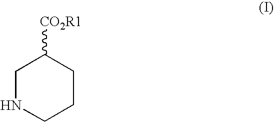

wherein R1 is methyl, ethyl, isopropyl, n-propyl, n-butyl, iso-butyl or tert-butyl; or -N(R2)2 where R2 is methyl, ethyl, n-propyl, isopropyl, n-butyl, isobutyl or tert-butyl. In a preferred embodiment, the nipecotate ester is ethyl nipecotate represented by Structural Formula II

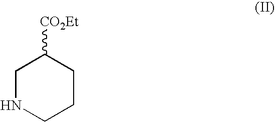

When resolving a racemic mixture of ethyl nipecotate according to a preferred embodiment of the invention, the resolving agent di-benzoyl-L-tartaric acid is reacted with ethyl nipecotate in a 91% aqueous 2B-ethanol solvent. The mixture is slowly heated until the ingredients are completely dissolved. The reaction is carried to completion, resulting in a mixture comprising diastereomeric di-benzoyl-L-tartrate salts. The reaction mixture is then allowed to slowly cool, preferably with seeding. As the mixture cools, the (S)-enantiomer-enriched tartrate salt precipitates from the reaction solution, while the (R)-enantiomer-enriched salt remains in solution. The (S)-enriched enantiomer salt may then be collected, washed and dried by conventional processes.

The reaction scheme for forming the (S)-enriched diastereomeric salt of ethyl nipecotate is illustrated in Scheme 1 below:

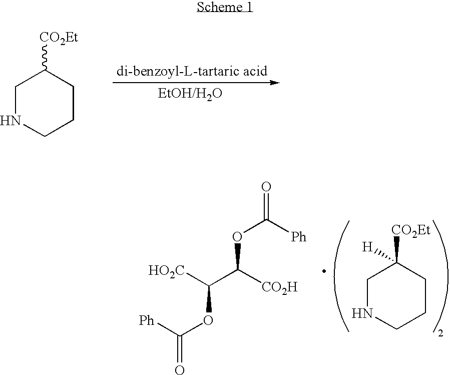

The diastereomeric (S) ethyl nipecotate salt of Scheme 1 may be recovered in high quality and at a high yield after a single crystallization or slurry operation. The (S) precipitate is shown by ¹H NMR to be a 2:1 nipecotate:tartrate complex as shown. Subsequent conversion of this tartrate precipitate to the corresponding free base and free acid, followed by Mosher amide formation and analysis, indicates the enantiomeric excess of the free amine to be ≧98%.

According to another feature of the invention, the yield of the desired enantiomer may be further increased by recycling the mother liquor remaining after the removal of the precipitate or slurry. The mother liquor consists substantially of the dissolved (R)-enriched diastereomeric salt in the solvent. In this recycling process, the (R)-enriched mother liquor is concentrated in vacuo, and the diastereomeric salt is converted to the (R)-enantiomer free base by conventional means, such as by reacting the salt with aqueous sodium carbonate. The enantiomer is separated from the concentrate by one or more extractions with a suitable aqueous organic solvent, such as methyl tert-butyl ether, and then concentrated. The concentrate is epimerized into the respective (R) and (S) enantiomers by dissolving it in a suitable solvent, such as 91% aqueous 2B-ethanol, and treating it with a suitable epimerization agent, such as catalytic sodium ethoxide. The racemic mixture is neutralized with an acid, such as concentrated hydrochloric acid, and filtered. The racemic mixture is thereafter resolved with an appropriate resolving agent as before, and the resulting precipitate is added to the precipitate from the initial resolution.

Sodium ethoxide is the preferred epimerization agent, although other known epimerization agents, such as sodium hydride, sodium methoxide, sodium or potassium tert-butoxide, potassium ethoxide and methoxide, various lithium salts and sodium amide, may also be utilized, provided that the epimerization agent does not promote competing reactions. Preferably, strong mineral acids, such as hydrochloric acid, sulfuric acid, nitric acid or phosphoric acid are used in the neutralization reaction, although the reaction may be carried out utilizing weaker acids. However, neutralization with weak acids may lead to inferior quality and/or tacky products in low yield. In the case of some weak acids, low yields may be caused by interferences from salts of such weak acids.

The reaction scheme for the recycling step is illustrated in Scheme 2 below:

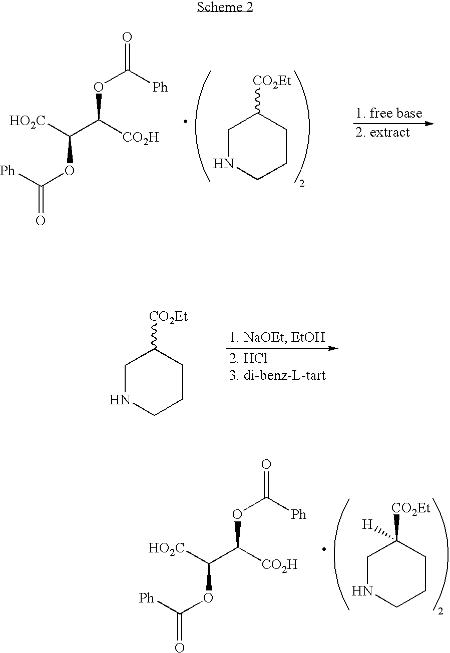

The recycling step may be repeated as many times as desired. owever, as will be appreciated by those skilled in the art, the benefits to be obtained by the recycling operation diminish with each successive repetition.

The resolution of racemic ethyl nipecotate by the process described above represents a straightforward, efficient and reproducible method for generating substantially pure resolved diastereomeric salts with high yield (35% or more, based upon 100% theory).

Although the preferred embodiment of the resolution described above utilizes di-benzoyl-L-tartaric acid as the resolving agent, other resolving agents, such as (S)-mandelic acid, may also be utilized. When (S)-mandelic acid is utilized in the reaction of Scheme 1, the reaction between racemic ethyl nipecotate and the resolving agent forms the (S)-mandelate salt as a 1:1 nipecotate:tartrate complex:

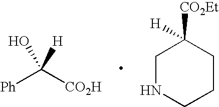

The degree of initial resolution obtained utilizing (S)-mandelic acid as the resolving agent is not as favorable as that obtained utilizing di-benzoyl-L-tartaric acid. Accordingly, when (S)-mandelic acid is utilized, it is preferred to utilize an extra crystallization step to obtain a substantially pure diastereomeric salt at high yield. Further details of this process are provided in Example 5.

Other optically active acids were tried as potential resolving agents. These acids include di-p-tolyl-L-tartaric acid, di-p-tolyl-D-tartaric acid, (R)-camphorsulfonic acid and (S)-camphorsulfonic acid. These acids either failed to form a crystalline salt, or in those cases in which a crystalline salt did form, recrystallization failed to provide an efficient optical purification. Thus, di-benzoyl-L-and D-tartaric acid and (S)- and (R)-mandelic acid are believed to be unique as readily available, efficient resolving agents for the compounds of Formula I.

A feature of the present invention is that the resolved diastereomeric tartrate or mandelate salts may be neutralized to afford the corresponding free base in situ, and thereafter directly reacted with other compounds to synthesize useful chiral compounds. There is no need to generate a free base (S)-enantiomer in a separate reaction step prior to formation of the chiral compound.

An example of the direct use of the di-benzoyl-L-tartrate salt of (S)-ethyl nipecotate of Scheme 1 in the preparation of a chiral amide compound is illustrated below in Scheme 3:

Scheme 3

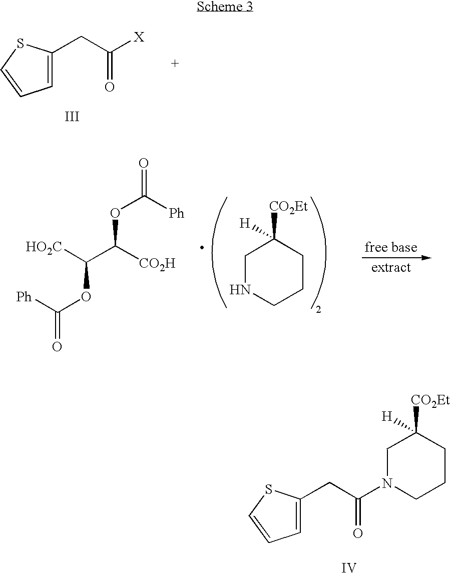

wherein X represents Cl, Br, F or OH.

In the reaction of Scheme 3, a precipitate or a slurry of the diastereomeric tartrate salt is reacted with a 2-thiopheneacetyl compound of Formula III to afford the amide compound of Formula IV. The coupling reaction between the tartrate salt and the 2-thiopheneacetyl compound enables the direct use of the tartrate salt in the formation of the end product of Formula IV, (S)-ethyl 1-(2-thiopheneacetyl)-3-piperidinecarboxylate. This product is believed to have pharmaceutical use as an immunopotentiating agent. In a preferred embodiment, the coupling reaction utilizes the acid chloride, 2-thiopheneacetyl chloride, under Schotten-Bauman conditions. The base used to neutralize the salts in situ also serves as the base for the Schotten-Bauman reaction. Alternatively, the corresponding bromide, fluoride or carboxylic acid (X=OH) may be utilized in the coupling reaction. The amide linkage fashioned by these procedures provides excellent yields of high quality product. Further details of an exemplary process for preparing the 3-piperidinecarboxylate product of Scheme III are provided in Example 3.

Although the resolution processes described above utilize either di-benzoyl-L-tartaric acid or (S)-mandelic acid as the resolving agent, thereby resulting in the formation of the respective (S)-enriched diastereomeric tartrate and mandelate salts, the reaction may alternatively be carried out utilizing either di-benzoyl-D-tartaric acid or (R)-mandelic acid as the resolving agent. In this event, the opposite diastereomeric salt is precipitated, namely the (R)-enriched tartrate or mandelate diastereomer.

The process described above is particularly useful for resolving a racemic mixture of ethyl nipecotate. In addition to ethyl nipecotate, related nipecotate esters such as methyl, isopropyl, n-propyl, n-butyl, iso-butyl and tert-butyl nipecotate may also be resolved.

A reaction scheme for forming the (S)-enriched diastereomeric tartrate salt of racemic N,N-dimethyl-3-carboxamide when di-benzoyl-L-tartaric acid is utilized as the resolving agent is illustrated below:

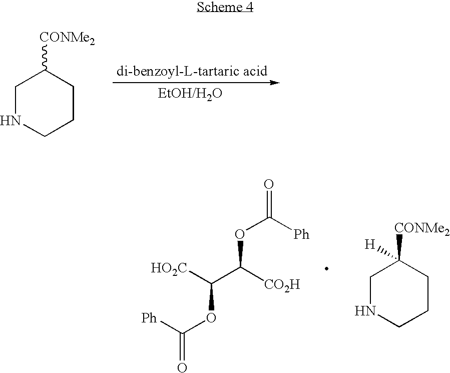

If desired, an extra crystallization step may be performed in order to improve the percentee. The di-benzoyl-L-tartrate salt may then be used in the formation of other chiral compounds. Example 6 describes details of the preparation of the (S)-N,N-dimethyl-3-piperidinecarboxamide-L-(dibenzoyl) tartrate salt shown above. This compound may be used as an intermediate in the preparation of the compound (S) N,N-dimethyl 1-benzyl-3-piperidinecarboxylate 1-methiodide, which is useful as an immunopotentiating agent. Details of the preparation of (S) N,N-dimethyl 1-benzyl-3-piperidinecarboxamide 1-methiodide) from the tartrate salt are provided in Examples 7-9.

Appropriate starting materials and reagents can be used to prepare the desired intermediates and compounds using the techniques described herein. The reagents are either commercially available, or may be prepared utilizing accepted chemical methods. The reaction time, reaction temperature and conditions described in the Examples below relate to the starting materials utilized in those Examples. The optimum time, temperature and conditions for any particular process are, as known in the chemical arts, frequently a compromise that is determined by considering the competing goals of throughput, which is generally favored by one set of reaction conditions, and maximum yield, which is generally favored by another set of conditions.

The following non-limiting examples are provided to more completely describe the process of the present invention. The examples also illustrate the preparation of inventive intermediates, as well as the use of intermediates in the formation of chiral compounds.

### EXAMPLE 1

### Preparation of (S)-di-benzoyl-L-tartrate Salt of Racemic Ethyl Nipecotate.

To a 3-neck, 5 L flask equipped with a heating mantle, mechanical stirrer, temperature probe, and reflux condenser topped with a calcium carbonate drying tube was charged 502 g (3.20 mol) of racemic (±) ethyl nipecotate, followed by 1005 mL of 91% aqueous 2B-ethanol. To the solution was added 286 g (0.80 mol) of di-benzoyl-L-tartaric acid as a slurry in 500 mL of 91% aqueous 2B-ethanol causing the temperature to rise to 37° C. Residual resolving agent was rinsed over with 505 mL of 91% ethanol. The mixture was then heated to dissolution, with complete dissolution observed at 76° C. The heat was turned off and the solution was allowed to gradually cool unaided. The solution was seeded at 71° C., and allowed to cool (precipitate observed at 63° C.) unaided to room temperature. The reaction mixture was allowed to stir a total of 18 hours after seeding. The white precipitate was collected and washed with 91% aqueous 2B-ethanol (2×200 mL) followed by vacuum drying at 45-50° C. for 6 hours to provide 346 g (32%) of the di-benzoyl-al-tartrate salt as a white solid: m.p. 173-175 ° C.; [α]−61.3° (c 1.22, MeOH), 99.1% ee for the free base; FTIR (KBr) 3428 (m), 2996 (m), 2854 (m), 2317 (w), 1721 (s), 1623 (s), 1454 (s), 1383 (s), 1269 (s), 1196 (s), 1121 (s), 715 (s) cm⁻¹; 500 MHz ¹H NMR (D₂O) δ7.96 (d, 4H, J=8.0 Hz), 7.55 (t, 2H, J=7.5 Hz), 7.42-7.37 (m, 4H), 5.55 (s, 2H), 4.09-3.97 (m, 4H), 3.27 (dd, 2H, J=13, 3.5 Hz), 3.11-3.00 (m, 4H), 2.88-2.80 (m, 2H), 2.77-2.63 (m, 2H), 1.94-1.90 (m, 2H), 1.72-1.52 (m, 6H), 1.08 (t, 6H, J=7.3 Hz); 125 MHz ¹³C NMR (D₂O) δ174.63, 173.58, 168.44, 134.66, 130.43, 129.52, 129.36, 75.87, 63.01, 44.71, 44.42, 38.77, 25.03, 21.23, 13.82. Analysis calculated: For C₃₄H₄₄N₂O₁₂: C, 60.69; H, 6.59; N, 4.18. Found: C, 60.58; H, 6.66; N, 4.27.

### EXAMPLE 2

### Epimerization/Resolution Recycle of (R)-enriched Salt.

To ca. 100 g (wet) of concentrated resolution filtrates and mother liquors was added 500 mL of MTBE followed by 500 mL of 15% sodium carbonate. After stirring mechanically for 1 hour to dissolve, the layers were separated and the aqueous layer was extracted with MTBE (1×500 mL, 2×250 mL). The combined organics were dried (Na₂SO₄), filtered, and concentrated in vacuo to give 27.0 g (58%) of (R)-enriched ethyl nipecotate (50% ee) as a yellow oil. To a solution of the above oil (10 g, 63.5 mmol) in 58 mL of aqueous 2B-ethanol was added 5.9 mL (15.9 mmol) of a 21 wt % solution of sodium ethoxide in ethanol. After refluxing for 1 hour, the mixture was cooled to room temperature and treated with concentrated HCl (1.31 mL, 15.9 mmol) and filtered through celite washing with 36 mL of 2B-ethanol. The combined filtrate and washings were treated with 11.29 g (31.7 mmol) of di-benzoyl-L-tartaric acid and the mixture was heated to reflux. Addition of 2 mL of water effected complete dissolution after which time the mixture was allowed to cool slowly. The mixture was seeded at 64° C., allowed to slowly cool to room temperature and stirred for a total of 16 hours after seeding. The crystals were collected and dried in vacuo at 45-50° C. to yield 7.30 g (35%) of dibenzoyl-L-tartrate salt as a white solid having ≧97% ee for the free base or the free amine.

### EXAMPLE 3

### Preparation of (S)-ethyl 1-(2-thiopheneacetyl)-3-piperidinecarboxylate.

To a 3-neck, 5 L flask equipped with a mechanical stirrer and a dropping funnel was charged 182 g (271 mmol) of the (S)-di-benzoyl-L-tartrate salt, followed by 590 mL of ethyl acetate. To the rapidly stirring slurry was added 715 mL of water followed by 482 mL (2.5 equivalents) of 15% sodium carbonate over 15 minutes via the dropping funnel. After stirring briefly, a solution of 2-thiopheneacetyl chloride (91.3 g, 568 mmol) (prepared from thiopheneacetic acid (Lancaster) and thionyl chloride according to the procedure described in Miller, W. H., Dessert, A. M., Anderson, G. W., J. Am. Chem. Soc. 1948, 70, 500) in 111 mL of ethyl acetate was added over 10 minutes via the dropping funnel, some gas evolution being observed. Upon completion of the addition, the mixture was allowed to stir for 30 minutes, at which time the layers were separated and the aqueous layer was washed with ethyl acetate (2×350 mL). The combined organics were dried (Na₂SO₄), filtered, and concentrated in vacuo to a yellow oil. Chromatography (1200 g of flash SiO₂, 1:1 then 1:3 hexanes:ethyl acetate) provided 150 g (99%) of (S)-ethyl 1-(2-thiopheneacetyl)-3-piperidinecarboxylate as a faintly off-white oil.

The (S)-ethyl 1-(2-thiopheneacetyl)-3-piperidinecarboxylate obtained via this reaction was a colorless to off-white oil in near quantitative yield and high purity after simple silica gel chromatography. Chromatography (8× loading) was used to remove the yellow color and the residual 2-thiophenacetic acid from the crude reaction mixture. This method routinely produced material of greater than 98% optical and chemical purity. Optical purity was determined by chiral capillary electrophoresis, and chemical purity was determined by ¹H NMR, HPLC and GC analysis.

### EXAMPLE 4

### Mosher Amide Formation for Chiral Analysis.

To a slurry of 55 mg of the (S)-di-benzoyl-L-tartrate salt of Example 1 in 1 ml of MTBE was added 200 μL of 15% sodium carbonate. After stirring to dissolve, the layers were separated and the aqueous layer was washed with MTBE (2×1 mL). The combined organics were dried (Na₂SO₄), filtered, and concentrated to a colorless oil (22 mg, 86%). The free base was dissolved in 220 μL of CH₂Cl₂ and treated sequentially with (R)-(+)-α-methoxy-α-(trifluoromethyl)phenylacetic acid ((R)-Mosher acid, 66 mg, 2 equivalents), 4-dimethylamino pyridine (2 mg, 0.1 equivalents), and 1,3-dicyclohexylcarbodiimide (58 mg, 2 equivalents) as a solution in 60 μL of CH₂Cl₂. After stirring for 30 minutes, the cloudy white reaction product was analyzed by GC and HPLC.

### EXAMPLE 5

### Preparation of (S)-mandelate Salt of Racemic Ethyl Nipecotate.

To a solution of racemic ethyl nipecotate (5.0 g, 32 mmol) in 40 mL of ethyl acetate was added (S)-mandelic acid (4.8 g, 32 mmol). The mixture was heated to 60° C. to dissolve all solids. Upon cooling to room temperature over 135 minutes and stirring an additional 60 minutes, the solid was collected via filtration and washed with 20 mL of ethyl acetate. Vacuum drying at room temperature for 17 hours produced 3.10 g (32%) of the title compound as a white solid (94% de as determined by the Mosher amide method). The solid from above was slurried in 24 mL of ethyl acetate and heated to dissolve. Slow cooling over 60 minutes to room temperature produced a thick slurry to which was added an additional 5 mL of ethyl acetate followed by stirring an additional 3 hours. Filtration, washing with 10 mL of ethyl acetate,and vacuum drying for 48 hours produced 2.65 g (27% overall) of the title compound as a white solid (99% de).

### EXAMPLE 6

### Preparation of (S)-N,N-dimethyl-3-piperidinecarboxamide-L-(dibenzoyl) Tartrate Salt.

To a solution of N,N-dimethyl-3-piperidinecarboxamide (4.32 g; 27.6 mmol;) in 43.2 ml of aqueous 2B-ethanol was added 4.95 g (13.8 mmol) di-benzoyl-L-tartaric acid. After heating to dissolve, the solvent was removed in vacuo leaving behind a white solid which was redissolved in 30.2 ml of 2B-ethanol with heating to reflux. Upon cooling to room temperature and stirring, the resultant white precipitate was collected via filtration and dried in vacuo to give (S)-N,N-dimethyl-3-piperidinecarboxamide-L-(dibenzoyl) tartrate salt in 38% yield (2.73 g) and 71% diastereomeric excess. This material was recrystallized from 27 ml of 99% 2B-ethanol by dissolving at reflux followed by cooling to room temperature and stirring for three hours. The white precipitate was collected by filtration and dried in vacuo to provide (S)-N,N-dimethyl-3-piperidinecarboxamide-L-(dibenzoyl) tartrate salt in 22% yield (1.55g) ,and 99% diastereomeric excess.

### EXAMPLE 7

### Preparation of (S)-N,N-dimethyl-3-piperidinecarboxamide.

(S)-N,N-dimethyl-3-piperidinecarboxamide-L-(dibenzoyl) tartrate salt (1.17 g; 2.28 mmol) from Example 6 was suspended in dichloromethane and shaken with 1N sodium hydroxide (6 ml). The dichloromethane was then dried with anhydrous potassium carbonate and evaporated under reduced pressure at ambient temperature, giving (S)-N,N-dimethyl-3-piperidinecarboxamide (275.3 mg) as a colorless oil, which was used without further purification. ¹H-NMR (CDCl₃) δ(ppm) 1.5 (m, 1H), 1.7 (m, 2H), 1.85 (m,1H), 2.65 (m, 2H), 2.85 (dd, 1H), 2.95 (s, 3H), 3.0 (m, 3H), 3.1 (s, 3H).

### EXAMPLE 8

### Preparation of (S)-N,N-dimethyl 1-benzyl-3-piperidinecarboxamide.

(S)-N,N-dimethyl-3-piperidinecarboxamide (275 mg; 1.76 mmol), diisopropylethylamine (310 L: 1.78 mmol), and benzyl bromide (210 L: 1.77 mmol) were dissolved in dichloromethane and the mixture was stirred at ambient temperature for about 20 hours. The mixture was then evaporated at ambient temperature under reduced pressure. The crude product was purified by chromatography on silica eluting with 5% methanol/95% dichloromethane, giving (S)-N,N-dimethyl 1-benzyl-3-piperidinecarboxamide (322.2 mg) as a colorless oil. MS m/z (positive ion) 247 (MH⁺; 100).

### EXAMPLE 9

### Preparation of (S)-N,N-dimethyl 1-benzyl-3-piperidinecarboxamide 1-methiodide.

(S)-N,N-dimethyl 1-benzyl-3-piperidinecarboxamide (295.2 mg; 1.2 mmol) was dissolved in diethyl ether (50 ml), and methyl iodide (2 ml) was added. The mixture was allowed to stir at ambient temperature for 3 days, and was then heated to reflux for 23 hours. The precipitate was filtered, washed with fresh diethyl ether, and then dried under reduced pressure at ambient temperature, giving (S)-N,N-dimethyl 1 benzyl-3-piperidinecarboxamide 1-methiodide (390.4 mg) as a pale yellow solid. MS m/z (positive ion) 261 (M⁺; 100).

In the foregoing discussion and examples, ¹H and ¹³C NMR spectra were recorded on an ARX-500 spectrometer. The optical purities (% ee) of the resolved ethyl nipecotate determined by Mosher amide analysis utilized capillary gas chromatography on a Hewlett-Packard model 5890 instrument equipped with a 25 m×0.25 mm 30M DB-1 column (initial temperature 60° C. ramped to 300° C. over 13 minutes, injector temperature 250° C.) with FID detection at 250° C., and by HPLC analysis on a Shimadzu SCL-10A instrument equipped with a 4.6 mm×250 mm Zorax SB-Phenyl column with gradient elution (1 mL/minute, acetonitrile-water, both with 0.05% TFA, 60% ACN ramped to 90% over 25 minutes) and detection at 220 nm. Absolute configuration of resolved ethyl nipecotate was determined through Mosher amide formation and comparison of retention times (GC and HPLC) with Mosher amides derived from commercially-obtained samples of (S)-ethyl nipecotate obtained from Chemie S.p.A., and samples of (R)-ethyl nipecotate prepared according to the procedure described in Zheng, X.; Day, C.; Gollamudi, R., Chirality, 1995, 7, 90.

Analysis of the chemical purity of (S)-ethyl 1-(2-thiopheneacetyl)-3-piperidinecarboxylate was conducted via the above GC method as well as by HPLC using a Shimadzu SCL-10A instrument equipped with a 4.6 mm×250 mm Zorbax SB-Phenyl column with isocratic elution (1 mL/min, acetonitrile-water both with 5% TFA, 40% ACN, 20 min) and detection at 220 nm. Optical rotations were measured with a Perkin-Elmer Model 241 polarimeter. Melting points were recorded on a Gallenkamp melting point apparatus and are uncorrected.

What is claimed is:

1. A process for resolving a racemic mixture of compounds of Structural Formula I 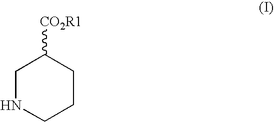
wherein R1 is C₁-C₄ alkyl or-N(R2)₂ where R2 is C₁-C₄ alkyl, comprising:
(a) reacting a racemic mixture comprising (R) and (S) enantiomers of a compound of Structural Formula I and a resolving agent selected from the group consisting of di-benzoyl-L-tartaric acid, di-benzoyl-D-tartaric acid, (S)-mandelic acid and (R)-mandelic acid in a solvent under conditions suitable for forming soluble diastereomeric salts comprising said (R) enantiomer and said resolving agent, and said (S) enantiomer and said resolving agent, respectively;
(b) controlling said reaction conditions such that said diastereomeric salt comprising one of said (R) and (S) enantiomers separates from the mixture, wherein the diastereomeric salt comprising the other of said enantiomers remains in solution; and
(c) isolating the separated salt from said mixture.

2. The process of claim 1, wherein the reaction takes place at from room temperature to reflux, and wherein the controlling step comprises cooling the reaction mixture to a temperature such that said diastereomeric salt comprising said one enantiomer precipitates out of solution and said diastereomeric salt comprising said other enantiomer remains in solution.

3. The process of claim 2, wherein R1 is ethyl, the resolving agent is di-benzoyl-L-tartaric acid and the diastereomeric salt precipitating from the mixture comprises the (S) enantiomer of ethyl nipecotate and di-benzoyl-L-tartrate.

4. A process for resolving racemic ethyl nipecotate, comprising:
(a) reacting a racemic mixture comprising (R) and (S) enantiomers of ethyl nipecotate with a resolving agent selected from the group consisting of di-benzoyl-L-tartaric acid and (S)-mandelic acid in a solvent at a temperature sufficient to form soluble diastereomeric salts comprising said (R) enantiomer and said resolving agent, and said (S) enantiomer and said resolving agent, respectively;
(b) cooling the reaction mixture to a temperature such that said diastereomeric salt comprising the (S) enantiomer precipitates from the mixture, wherein the diastereomeric salt comprising the (R) enantiomer remains in solution; and
(c) isolating the precipitated salt comprising the (S) enantiomer from said mixture.

5. The process of claim 4, wherein the resolving agent is di-benzoyl-L-tartaric acid.

6. The process of claim 5, wherein the solvent is selected from the group consisting of ethyl acetate, isopropyl acetate, butyl acetate, acetone, acetonitrile, methyl tert-butyl ether, tetrahydrofuran, 1,4-dioxane, diethyl ether, C1 to C4 alcohols, toluene, water, and mixtures of the foregoing.

7. The process of claim 6, wherein the solvent comprises ethanol.

8. The process of claim 5, further comprising:
(d) concentrating the reaction mixture remaining after isolation of the precipitated salt, said remaining mixture including the diastereomeric salt comprising the (R) enantiomer;
(e) reacting the concentrated reaction mixture comprising the (R) enantiomer with a base to free the (R) enantiomer;
(f) separating the (R) enantiomer from the mixture;
(g) epimerizing said (R) enantiomer in a reaction solution, and neutralizing said reaction solution; and
(h) resolving said reaction solution and isolating the precipitated salt comprising the (S) enantiomer as described in (a), (b) and (c).

9. The process of claim 8, wherein the reaction solution of (9) is neutralized with a mineral acid selected from the group consisting of hydrochloric acid, sulfuric acid, nitric acid and phosphoric acid.

10. The process of claim 4, wherein the resolving agent is (S)-mandelic acid.

11. A compound of the formula

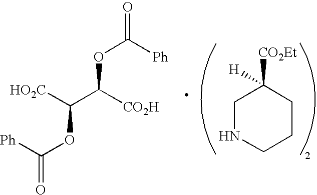

12. A compound of the formula

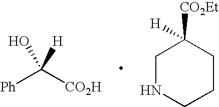

13. A compound of the formula

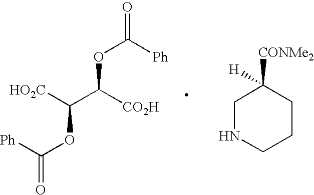

14. A process for preparing a compound of the formula 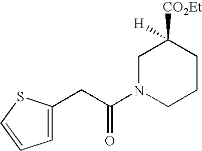
comprising:
reacting a compound of the formula 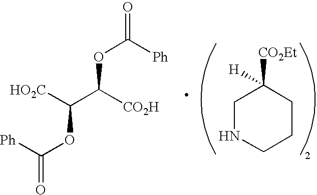
with a compound of the formula 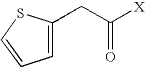
wherein X is Cl, Br, F or OH.

15. The process of claim 14, wherein X is Cl.

16. The process of claim 15, wherein the compound of the formula 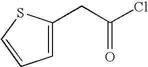
is prepared by reacting thiopheneacetic acid with thionyl chloride.

17. The process of claim 16, wherein the reaction is carried out in the presence of a base.

18. The process of claim 17, wherein the base is sodium carbonate.

19. The process of claim 14, wherein the compound 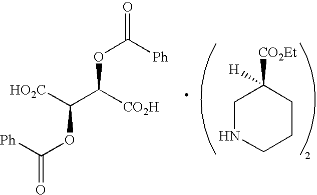
is prepared by:
reacting a racemic mixture comprising (R) and (S) enantiomers of ethyl nipecotate with di-benzoyl-L-tartaric acid in a solvent to form soluble diastereomeric salts comprising said (R) enantiomer and said di-benzoyl-L-tartaric acid, and diastereomeric salts comprising said (S) enantiomer and said di-benzoyl-L-tartaric acid, respectively;
cooling the reaction mixture to a temperature such that said diastereomeric salt comprising said (S) enantiomer separates from the mixture, wherein the diastereomeric salt comprising said (R) enantiomer remains in solution; and
isolating the diastereomeric salt comprising said (S) enantiomer from said mixture.
